(19) United States
(12) Patent Application Publication
(10) Pub. No.: US 2022/0183921 A1
(43) Pub. Date: Jun. 16, 2022

(54) Wall-Mounted Back Care Apparatus

(71) Applicant: Antonia Scott, Waco, TX (US)

(72) Inventor: Antonia Scott, Waco, TX (US)

(21) Appl. No.: 17/122,189

(22) Filed: Dec. 15, 2020

(51) Int. Cl. A61H 7/00; A61B 17/32; A61M 35/00; A47K 7/02

(52) U.S. Cl. CPC A61H 7/002; A61B 17/32; A61M 35/00; A47K 7/024; A61B 2017/320004; A61B 2017/00862; A61H 2205/081; A61H 2201/0126; A61M 2209/082; A61M 2205/0216; A61M 2210/10

(57) ABSTRACT

A wall-mounted back care apparatus for handsfree washing, lotion application, and scratching includes a pair of mounting brackets. Each mounting bracket has a mounting plate and a receiver coupled to the mounting plate. The mounting plate is mounted to a wall of a shower or a bathroom. A mounting rod is coupled to the pair of mounting brackets and is selectively engageable within the receiver of each mounting bracket. A backboard is coupled to the mounting rod. The backboard has a concave front face and a convex rear face. A cover is coupled to the backboard. The cover is selectively engageable with the backboard to be continuously disposed over the front face of the backboard. The cover exfoliates skin, applies soap or lotion, or scratches an itch.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### REGARDING PRIOR DISCLOSURES BY THE INVENTOR OR JOINT INVENTOR

Not Applicable

### BACKGROUND OF THE INVENTION

### (1) Field of the Invention

The disclosure relates to back care devices and more particularly pertains to a new back care device for handsfree washing, lotion application, and scratching.

### (2) Description of Related Art Including Information Disclosed Under 37 CFR 1.97 and 1.98

The prior art relates to back care devices. Known devices often incorporate fixed scrubbers rather than a removable and replaceable cover. Most known devices lack an angle adjustment. Existing back care devices are typically planar and do not match the contours of the user's back.

### BRIEF SUMMARY OF THE INVENTION

An embodiment of the disclosure meets the needs presented above by generally comprising a pair of mounting brackets. Each mounting bracket has a mounting plate and a receiver coupled to the mounting plate. The mounting plate is configured to be mounted to a wall of a shower or a bathroom. A mounting rod is coupled to the pair of mounting brackets and is selectively engageable within the receiver of each mounting bracket. A backboard is coupled to the mounting rod. The backboard has a concave front face and a convex rear face. A cover is coupled to the backboard. The cover is selectively engageable with the backboard to be continuously disposed over the front face of the backboard. The cover is configured to exfoliate skin, apply soap or lotion, or scratch an itch.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a wall-mounted back care apparatus according to an embodiment of the disclosure.

FIG. 2 is an isometric exploded view of an embodiment of the disclosure.

FIG. 3 is an isometric exploded view of an embodiment of the disclosure.

FIG. 4 is a front elevation view of an embodiment of the disclosure.

FIG. 5 is a bottom plan view of an embodiment of the disclosure.

FIG. 6 is an in-use view of an embodiment of the disclosure.

### DETAILED DESCRIPTION OF THE INVENTION

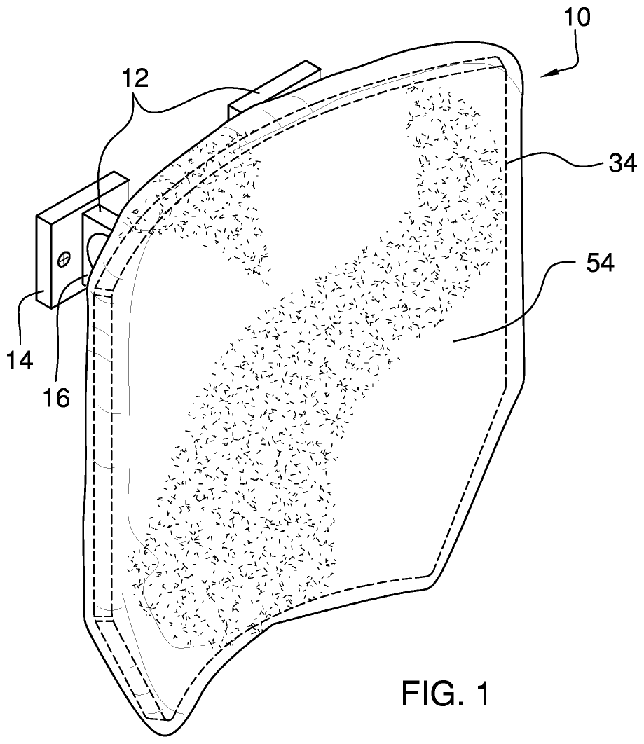

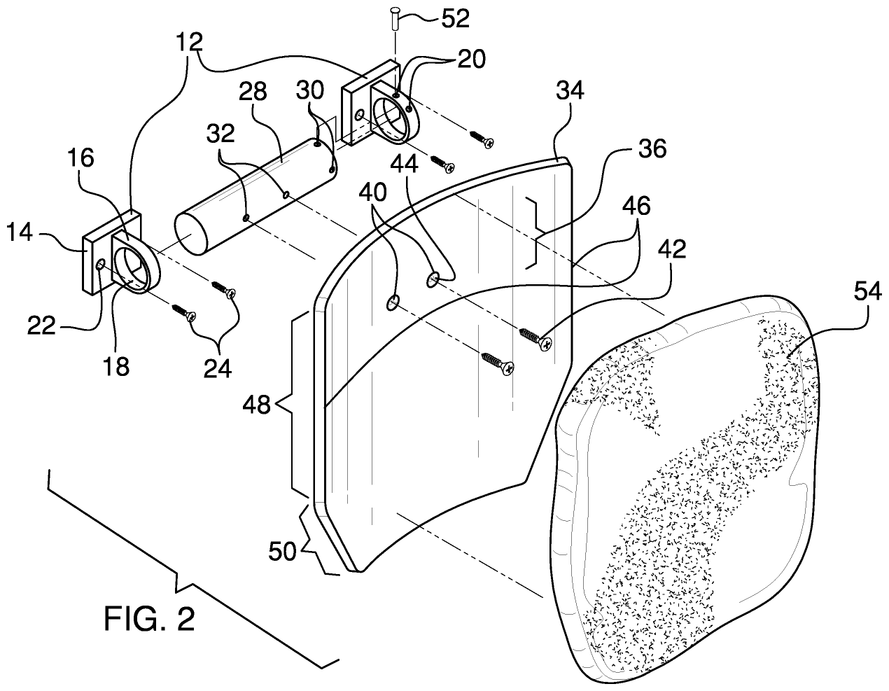

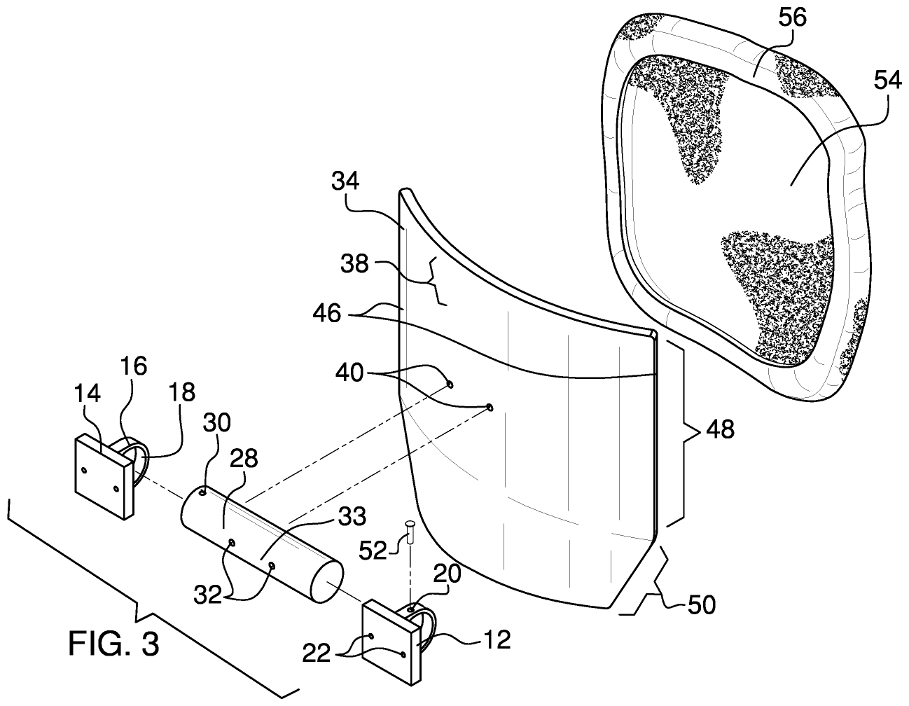

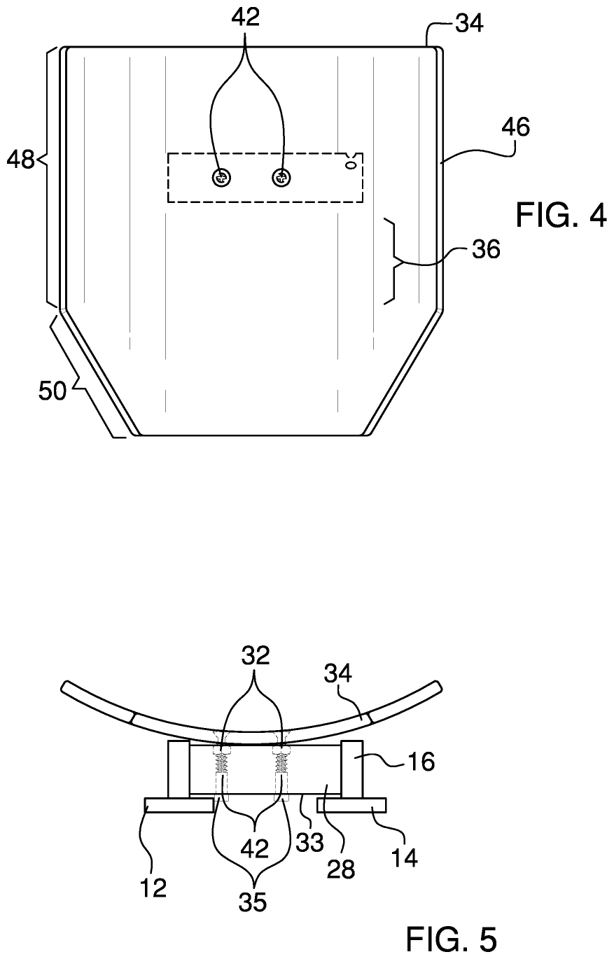

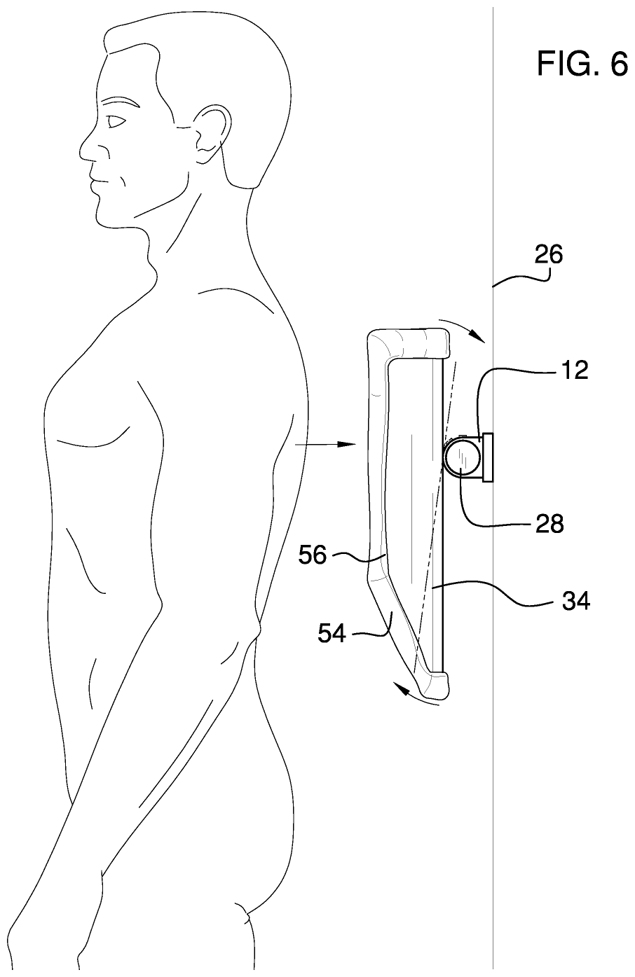

With reference now to the drawings, and in particular to FIGS. 1 through 6 thereof, a new back care device embodying the principles and concepts of an embodiment of the disclosure and generally designated by the reference numeral 10 will be described.

As best illustrated in FIGS. 1 through 6, the wall-mounted back care apparatus 10 generally comprises a pair of mounting brackets 12. Each mounting bracket 12 has a mounting plate 14 and a receiver 16 coupled to the mounting plate 14. The receiver 16 may extend perpendicularly from the mounting plate 12 and has a rod aperture 18 extending therethrough. The receiver 16 may be rounded to conform with the rod aperture 18. The receiver 16 of at least one of the mounting brackets 12 has a pair of first position apertures 20 extending through to the rod aperture 18. Each mounting plate 14 has a pair of mounting holes 22 extending therethrough to receive a pair of mounting screws 24. The mounting plate 14 is configured to be mounted to a wall 26 of a shower or a bathroom.

A mounting rod 28 is coupled to the pair of mounting brackets 12. The mounting rod 28 is selectively engageable within the receiver 16 of each mounting bracket. The mounting rod 28 has a pair of second position apertures 30 corresponding with the pair of first position apertures 20. The mounting rod 28 has a pair of attachment apertures 32 extending therethrough. There may be a pair of slots on a rod backside 33 of the mounting rod to accommodate a pair of nuts 35.

A backboard 34 is coupled to the mounting rod 28. The backboard 34 has a concave front face 36 and a convex rear face 38. The backboard 34 has a pair of attachment holes 40 extending from the front face 36 through the convex rear face 38 and aligning with the pair of attachment apertures 32 to receive a pair of attachment bolts 42. Each attachment hole 40 may have a countersunk portion 44 in the front face 36 of the backboard to prevent the user from feeling the attachment bolts 42. The backboard 34 has a pair of side edges 46. Each side edge 46 may have a vertical portion 48 and a tapered portion 50.

A locking pin 52 is selectively engageable through the first position apertures 20 and the second position apertures 30 to secure the backboard 34 in a vertical position or a tilted position. The rounded nature of the receiver 16 of each mounting bracket prevents interference with the backboard 34.

A cover 54 is coupled to the backboard 34. The cover 54 is selectively engageable with the backboard 34 to be continuously disposed over the front face 36 of the backboard. The cover 54 may have an elastic perimeter portion 56 extending on to the convex rear face 38 of the backboard to secure the cover 54. The cover 54 is configured to exfoliate skin, apply soap or lotion, or scratch an itch.

In use, the user adjusts the locking pin 52 to create the desired orientation of the backboard 34. The user then places his or her back against the front face 36 of the backboard to scratch, exfoliate, or apply soap or lotion.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A wall-mounted back care apparatus comprising:
a pair of mounting brackets, each mounting bracket having a mounting plate and a receiver coupled to the mounting plate, the mounting plate being configured to be mounted to a wall of a shower or a bathroom;
a mounting rod coupled to the pair of mounting brackets, the mounting rod being selectively engageable within the receiver of each mounting bracket;
a backboard coupled to the mounting rod, the backboard having a concave front face and a convex rear face; and
a cover coupled to the backboard, the cover being selectively engageable with the backboard to be continuously disposed over the front face of the backboard, the cover being configured to exfoliate skin, apply soap or lotion, or scratch an itch.

2. The wall-mounted back care apparatus of claim 1 further comprising each mounting plate having a pair of mounting holes extending therethrough to receive a pair of mounting screws.

3. The wall-mounted back care apparatus of claim 1 further comprising the receiver extending perpendicularly from the mounting plate and having a rod aperture extending therethrough.

4. The wall-mounted back care apparatus of claim 3 further comprising the receiver being rounded to conform with the rod aperture.

5. The wall-mounted back care apparatus of claim 4 further comprising the receiver of at least one of the mounting brackets having a pair of first position apertures extending through to the rod aperture; the mounting rod having a pair of second position apertures corresponding with the pair of first position apertures; a locking pin being selectively engageable through the first position apertures and the second position apertures to secure the backboard in a vertical position or a tilted position.

6. The wall-mounted back care apparatus of claim 1 further comprising the mounting rod having a pair of attachment apertures; the backboard having a pair of attachment holes extending from the front face through the convex rear face and aligning with the pair of attachment apertures to receive a pair of attachment bolts.

7. The wall-mounted back care apparatus of claim 1 further comprising each attachment hole having a countersunk portion in the front face of the backboard.

8. The wall-mounted back care apparatus of claim 1 further comprising the backboard having a pair of side edges, each side edge having a vertical portion and a tapered portion.

9. The wall-mounted back care apparatus of claim 1 further comprising the cover having an elastic perimeter portion, the elastic perimeter portion extending on to the convex rear face of the backboard to secure the cover.

10. A wall-mounted back care apparatus comprising:
a pair of mounting brackets, each mounting bracket having a mounting plate and a receiver coupled to the mounting plate, the receiver extending perpendicularly from the mounting plate and having a rod aperture extending therethrough, the receiver being rounded to conform with the rod aperture, the receiver of at least one of the mounting brackets having a pair of first position apertures extending through to the rod aperture, each mounting plate having a pair of mounting holes extending therethrough to receive a pair of mounting screws, the mounting plate being configured to be mounted to a wall of a shower or a bathroom;
a mounting rod coupled to the pair of mounting brackets, the mounting rod being selectively engageable within the receiver of each mounting bracket, the mounting rod having a pair of second position apertures corresponding with the pair of first position apertures, the mounting rod having a pair of attachment apertures;
a backboard coupled to the mounting rod, the backboard having a concave front face and a convex rear face, the backboard having a pair of attachment holes extending from the front face through the convex rear face and aligning with the pair of attachment apertures to receive a pair of attachment bolts, each attachment hole having a countersunk portion in the front face of the backboard, the backboard having a pair of side edges, each side edge having a vertical portion and a tapered portion;
a locking pin being selectively engageable through the first position apertures and the second position apertures to secure the backboard in a vertical position or a tilted position; and
a cover coupled to the backboard, the cover being selectively engageable with the backboard to be continuously disposed over the front face of the backboard, the cover having an elastic perimeter portion, the elastic perimeter portion extending on to the convex rear face of the backboard to secure the cover, the cover being configured to exfoliate skin, apply soap or lotion, or scratch an itch.
